(19) United States
(12) Patent Application Publication
(10) Pub. No.: US 2023/0104282 A1
(43) Pub. Date: Apr. 6, 2023

(54) Undenatured Type II Collagen in Animal Food and Treats

(71) Applicant: Lonza Greenwood LLC, Greenwood, SC (US)

(72) Inventors: Vijaya Juturu, North Bruswick, NJ (US); Kevin Owen, Dover, NH (US); Tyler White, Greenwood, SC (US)

(21) Appl. No.: 17/908,007

(22) PCT Filed: Mar. 23, 2021
(86) PCT No.: PCT/US2021/023656

§ 371 Date: Aug. 30, 2022

Related U.S. Application Data
(60) Provisional application No. 62/993,875, filed on Mar. 24, 2020.

(51) Int. Cl. A23K 20/147; A23K 40/25; A23K 40/20; A23K 50/42; A61K 9/00; A61K 38/39

(52) U.S. Cl. CPC A23K 20/147; A23K 40/25; A23K 40/20; A23K 50/42; A61K 9/0056; A61K 38/39

(57) ABSTRACT

The present disclosure is directed to processed animal food and/or treat that includes undenatured type II collagen. The undenatured type II collagen is introduced to the animal food and/or treat prior to processing, and at least 30% or more of the undenatured collagen remains undenatured in the after processing the animal food and/or treat.

### BACKGROUND

In recent years, the use of collagen to treat various conditions has become exceedingly popular. Collagen is a protein that can be found in muscles, bones, skin, blood vessels, and in other parts of the body. There are various different types of collagen depending upon its function and form. For instance, Type I collagen, the most abundant collagen, is made of fibers found in tendons, ligaments, organs and skin. Type II collagen, on the other hand, primarily helps build cartilage, a major structural entity that sits on the surfaces of those bones which comprise articulating joints. Type III collagen is a major component of the extracellular matrix that makes up organs and skin. Type III collagen also forms blood vessels and tissue within the heart. Type IV collagen is found primarily in the skin as sheet-like structures in the cutaneous basal lamina. Furthermore, Collagen peptides are portions of one or more of the a strands of any type of collagen formed through enzymatic hydrolysis of collagen. Collagen peptides are often used in beverages and food products, as they are water-soluble and non-gelling.

However, collagen production in the body of most mammals tends to slow as the mammal ages. For instance, many mammals, including household animals and livestock suffer from age related arthritis as well as exercise induced joint pain, muscle pain, cartilage loss, and bone loss. This can be detrimental to the function of the animal in competition or working settings, and can also result in loss of quality of life for household pets.

Collagen has been found to effectively treat arthritis and other joint pain in mammals. For example, U.S. Pat. No. 9,066,926 discloses a method of reducing exercise-induced joint pain in mammals by administering to a mammal Type II collagen. This patent also discloses the mechanism of action through which this ingredient operates: oral tolerance. This putative mechanism entails the stimulation of T regulatory cells (Treg), located in gut associated lymphatic tissue, to specifically recognize antigenic determinants (epitopes) on the native collagen protein. Once induced, the Tregs exit the gut area and migrate to the joint space where they stimulate chondrocytes to lay down new Type II collagen thereby enhancing the structural integrity and flexibility of the articulating joint. One such example, for which clinical data has been published is the knee. The '926 patent is incorporated herein by reference.

However, so far, undenatured collagen sources have only been available in “raw” form, meaning that the undenatured collagen has not been incorporated into a processed animal food or treat, as undenatured collagen is sensitive to high temperatures, mechanical processing, and changes in pH. Particularly, it was believed that to form a product containing undenatured collagen, the product could not undergo cooking, such as baking, frying, or otherwise being heated, including incorporation with heated liquids or steams, as it would cause the collagen to denature. Further, it was previously taught and believed that undenatured collagen would denature when exposed to acidic conditions, and was therefore included in compositions having a pH of greater than 7. Moreover, even mechanical processing, such as pressing and extrusion was believed to negatively impact amounts of undenatured collagen in processed animal food and treats

Therefore, currently available undenatured collagen products include powders and capsules, that can be optionally incorporated into final products that do not require heating or acidic conditions, or instead, directly consumed. This has been particularly problematic for administration to non-human mammals, such as pets and livestock, as supplements and capsules sink to the bottom of bowls of foods or liquids when added over food or water as a dry powder, and remain unconsumed by the mammal, or, in the case of capsules, can be difficult, if not impossible to administer to the mammal. Therefore, many face problems in administering undenatured collagen to mammals, such as pets and livestock, as they are unwilling to voluntarily consume the supplement or capsule.

Although collagen can offer various advantages when administered to a mammal, a need exists for a processed animal food and/or treat that contains undenatured collagen. It would be a further benefit to provide a processed animal food and/or treat that had a high recovery rate of undenatured collagen as compared to the pre-processed food and/or treat. A need also exists for a processed animal food and/or treat containing undenatured collagen to support healthy mammals. Furthermore, it would be a benefit to provide a processed animal food and/or treat containing undenatured collagen for supporting trained mammals.

### SUMMARY

In general, the present disclosure is directed to a processed animal food and/or treat composition that includes an undenatured type II collagen after processing at a temperature of about 37° C. or greater. In one aspect, the composition is a processed animal food composition. Additionally or alternatively, in an aspect, the composition is a processed animal treat or chew.

In one aspect, the undenatured type II collagen is incorporated into the processed animal food and/or treat composition as part of a collagen composition that includes one or more different types of collagen in addition to the undenatured type II collagen. In an aspect the one or more different types of collagen include native type II collagen, collagen peptides, or a mixture thereof.

In a further aspect, an amount of undenatured type II collagen in incorporated into the composition prior to processing, and at least about 30% or more of the undenatured type II collagen is recovered after processing. In one aspect, 45% or more of the undenatured type II collagen is recovered after processing. Additionally or alternatively, in an aspect, 60% or more of the undenatured type II collagen is recovered after processing. Furthermore, in one aspect, 85% or more of the undenatured type II collagen is recovered after processing.

In another aspect, the processed animal food and/or treat undergoes processing that includes withstanding a temperature of about 40° C. or greater. Furthermore, in one aspect, the processing lasts from 6 seconds to about 2 hours.

In one aspect, the animal food and/or treat includes one or more of a protein source, a grain, a flavoring, or a coloring.

The present disclosure is also generally directed to a method of forming a processed animal food and/or treat. The method includes combining an undenatured type II collagen with at least one animal food and/or treat component and processing the undenatured type II collagen and at least one animal food and/or treat component at a temperature of about 37° C. or greater, where at least about 30% or more of the undenatured type II collagen is recovered in the processed animal food and/or treat after processing as compared to the amount of undenatured type II collagen prior to processing.

In one aspect, the processing includes subjecting the undenatured type II collagen and at least one animal food and/or treat component to a temperature of about 40° C. for at least about 10 minutes. In a further aspect, the processing includes subjecting the undenatured type II collagen and at least one animal food and/or treat component to a temperature of about 100° C. for at least about 1 minute. Furthermore, in one aspect, the processing includes subjecting the undenatured type II collagen and at least one animal food and/or treat component to a temperature of about 120° C. for at least about 1 minute. In one aspect, the processing further includes extruding the composition. Moreover, in one aspect, the processing further comprises injection molding the composition. In a further aspect, the processing includes pelletizing the undenatured type II collagen and at least one animal food and/or treat component.

Nonetheless, the present disclosure also generally includes a method of improving one or more of joint health, muscle health, bone health, skin health, or fitness comprising, administering to a non-human mammal an effective amount of a processed animal food and/or treat according to the present disclosure and/or any one or more of the above discussed aspects.

### Definitions

As used herein, the terms “about,” “approximately,” or “generally,” when used to modify a value, indicates that the value can be raised or lowered by 10% and remain within the disclosed aspect.

The term “therapeutically effective amount” as used herein, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “nutraceutical” and refers to any compound added to a dietary source (e.g., a food, beverage, or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved joint health, resilience, and recovery, and can include without limitation canine, equine, feline, bovine, ovine, or porcine mammals. For purposes of this application, “mammal” does not include human subjects, and may be used interchangeably with anima.

As used herein, “healthy” refers to the absence of illness or injury.

The term “physical activity” means activity that lasts about 10 minutes or more, such as about 25 minutes or more, such as about 30 minutes or more, such as at least about 45 minutes or more, and where the heart rate of the mammal reaches about 30% to about 85% of its maximum heart rate, such as about 40% to about 80%, such as about 50% to about 75% of the maximum heart rate of the mammal.

The term “intensive physical activity” means activity that lasts about 20 minutes or more, such as about 25 minutes or more, such as about 30 minutes or more, such as at least about 45 minutes or more, and where the heart rate of the mammal reaches about 50% to about 99% of its maximum heart rate, such as about 55% to about 95%, such as about 60% to about 90%, such as about 705 to about 85% of the maximum heart rate of the mammal.

Unless otherwise noted, “collagen” as used herein refers to all forms of collagen, either with or without denaturation, without or without salts or stabilizing agents, and fibrillar and non-fibrillar types of collagen not limited to fibril associated collagens with interrupted triple helices (FACIT, Type IX, XII, XIV, XIX, XXI), including short chain collagen (generally Types VII and X), basement membrane (Type IV), Multiplexin (multiple triple helix domains with interruptions (Type XV, XVIII), and other types of collagen (Types VI, VII).

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to a processed animal food and/or treat, where the processing includes temperatures of about 37° C. or greater, that contains undenatured collagen, such as, in one aspect, an undenatured type II collagen. Particularly, the present disclosure has found the an undenatured collagen that has been carefully formed to preserve the epitopes on the undenatured strands can be used to form a processed animal food and/or treat, even when the processed animal food and/or treat requires high temperature processing, high pressure processing, mechanical processing, high moisture content, and/or low pH levels for production and/or storage.

For instance, an undenatured collagen according to the present disclosure may be included in a processed animal food and/or treat that has undergone processing that includes a temperature of about 37° C. or greater, such as about 40° C. or greater, such as about 45° C. or greater, such as about 50° C. or greater, such as about 55° C. or greater, such as about 60° C. or greater, such as about 65° C. or greater, such as about 70° C. or greater, such as about 75° C. or greater, such as about 80° C. or greater, such as about 85° C. or greater, such as about 90° C. or greater, such as about 95° C. or greater, such as about 100° C. or grater, such as about 105° C. or greater, such as about 110° C. or greater, such as about 120° C. or greater, such as about 130° C. or greater, such as about 140° C. or greater, such as about 150° C. or greater, such as about 160° C. or greater, such as about 170° C. or greater, such as about 180° C. or greater, such as about 190° C. or greater, such as about 200° C. or greater, such as up to about 300° C. or less, such as about 275° C. or less, such as about 250° C. or less.

In one aspect, the processed animal food and/or treat processed according to one or more of the above temperatures may undergo processing for a time of about 3 seconds or more, such as about 6 seconds or more, such as about 1 minute or more, such as about 1.5 minutes or more, such as about 2 minutes or more, such as about 5 minutes or more, such as about 10 minutes or more, such as about 15 minutes or more, such as about 20 minutes or more, such as about 30 minutes or more, such as about 1 hour or more, such as about 1.5 hours or more, such as about 2 hours or more, such as, in one aspect, up to about 4 hours, such as about 5 hours or less, such as about 4 hours or less, such as about 3 hours or less, such as about 2 hours or less, such as about 1 hour or less, such as about 30 minutes or less, or any ranges or values therebetween.

Additionally or alternatively, the processed animal food and/or treat may undergo any one or more of the above mentioned temperatures or times, and may also undergo a high pressure process, either at the same time as the high temperature processing, or before or after the high temperature processing. In such an aspect, the processed animal food and/or treat may undergo a high pressure process of about 50 psi or greater, such as about 100 psi or greater, such as about 200 psi or greater, such as about 300 psi or greater, such as about 400 psi or greater, such as about 500 psi or greater, such as about 600 psi or greater, such as about 700 psi or greater, such as about 800 psi or greater, such as about 900 psi or greater, such as about 1000 psi or greater, such as about 1100 psi or greater, such as about 1200 psi or greater, such as about 1300 psi or greater, such as about 1400 psi or greater, such as about 1500 psi or greater, such as about 1600 psi or greater, such as about 1700 psi or greater, such as about 1800 psi or greater, such as about 1900 psi or greater, such as about 2000 psi or greater, up to about 300 psi or less.

For instance, in one aspect, the high pressure may additionally or alternatively be mechanical pressure such as extrusion, molding, pressing, punching, pulling, pelletizing or the like. In one aspect, the mechanical pressure may be injection molding or pressure molding, or a combination thereof. Furthermore, in an aspect, the mechanical pressure may be extrusion. Nonetheless, in one aspect, the mechanical pressure may be in addition to any one or more of the other processing methods discussed herein.

In another aspect, processing may occur at a pH of about 2.5 to about 7, such as about 3 to about 6, such as about 3.25 to about 5, such as about 3.5 to about 4.5. Of course, in one such aspect, the low pH processing may be in regards to beverage processing, however, one or more processed foods may also undergo low pH processing.

Additionally or alternatively, in one aspect, the processed animal food and/or treat may have a high moisture content either before processing, after processing, or both before and after processing, and still maintain high levels of undenatured collagen. For instance, in one aspect, the processed animal food and/or treat may have a moisture content of about 5% or greater, such as about 10% or greater, such as about 15% or greater, such as about 20% or greater, such as about 25% or greater, such as about 30% or greater, or any ranges or values therebetween. In one aspect, the animal food and/or treat may initially have a moisture content according to the above, and may withstanding being dried while maintaining good recovery of undenatured collagen.

Furthermore, notwithstanding the processing conditions selected, the processing may include, in one aspect, baking, frying, steaming, boiling, autoclaving, or otherwise heating, cooking, or sterilizing the processed animal food and/or treat. Additionally or alternatively, processing may include a mechanical mixing process, such as emulsifying, shearing, gelling, homogenizing, or other mixing and/or incorporation processes known in the art.

In another aspect, the present disclosure has found that the processed animal food and/or treat may also include on or more further ingredients, and that the collagen remains undenatured according to the below mentioned recovery rates. For instance, in one aspect, the processed animal food and/or treat may include a sweetener, a preservative, a spice, a coloring, a dye, a plant protein, a fruit component, a flavoring, or other animal food and/or treat component as known in the art, or combinations thereof. For instance, in one aspect, the processed animal food and/or treat may include a sweetener such as sugar or an artificial sweetener, or may contain a sweetener syrup. Furthermore, in one aspect, the processed animal food and/or treat may include one or more fruit components, such as fruit juice or juices. Particularly, it was found that citrus components may be used, even though they posses a relatively low pH, and the undenatured collagen may be recovered as discussed above. Of course, other animal food and/or treat components may be used as known in the art, including gluten free flours and components in addition to traditional flours and sweeteners.

Regardless of the processing conditions selected, undenatured type II collagen can be recovered from an animal food and/or treat according to the present disclosure post processing, such that at least about 30% or more of the undenatured collagen is recovered in the processed animal food and/or treat after processing as compared to the amount of undenatured collagen pre-processing, such as about 35% or more, such as about 40% or more, such as about 45% or more, such as about 50% or more, such as about 55% or more, such as about 60% or more, such as about 65% or more, such as about 70% or more, such as about 75% or more, such as about 80% or more, such as about 85% or more, such as about 90% or more, such as about 95% or more of the undenatured collagen remains in the processed animal food and/or treat as compared to the amount of undenatured collagen contained or added to the pre-processed animal food and/or treat. Thus, in one aspect, an amount of undenatured collagen may be recovered from the processed animal food and/or treat after processing, according to the above percentages.

In one aspect, the undenatured collagen according to the present disclosure is incorporated into the processed animal food and/or treat as a collagen composition. The collagen composition may include one or more of any collagen as defined above, and/or, in one aspect, may include one or more of Type I collagen, Type II collagen, Type III collagen, Type IV collagen, or collagen peptides, or a mixture thereof. In one aspect, the collagen composition contains Type II collagen alone or in combination with one or more of Type I collagen, Type III collagen, Type IV collagen, or collagen peptides. In one aspect, the collagen composition may include a mixture of type II collagen (sometimes referred to as native type II collagen) and undenatured type II collagen. Additionally or alternatively, the collagen composition may include a mixture of native type II collagen and undenatured type II collagen, in addition to a further collagen, such as Type I, Type III, Type IV, or collagen peptides.

As indicated above, in one aspect, the processed animal food and/or treat contains a collagen composition, particularly a Type II collagen composition such as an undenatured Type II collagen composition. Type II collagen for use in the present disclosure can be obtained from any suitable source. For instance, the collagen can be derived from a variety of mammalian sources, avian sources, or can be obtained from various fish species or a combination thereof. For instance, the collagen can be obtained from salmon, shark, poultry, porcine, eggshells, turkey cartilage, bovine cartilage, and the like. In one embodiment, for instance, the Type II collagen can be obtained as disclosed in U.S. Pat. No. 7,083,820 to Schilling which is incorporated by reference. For example, undenatured Type II collagen is available commercially as UC-II® brand from LONZA Consumer Health Inc. UC-II® brand is a natural ingredient that contains a glycosylated, undenatured Type II collagen. The collagen composition can also comprise a hydrolyzed collagen. The collagen composition can also comprise a pure protein or active peptide fragments. In one embodiment, the collagen composition can be free of any bone or bone material. In other embodiments, the collagen composition can be free of any transforming growth factors (TGFs), bone morphogenetic proteins (BMPs), or both. In still another embodiment, the collagen composition comprises Type II collagen and is completely free of any Type I collagen.

In preparing animal tissue for oral administration, in one embodiment, the Type II collagen containing tissue can be first dissected free of surrounding tissues and diced or otherwise comminuted into particles. The particulate, or milled, cartilage can be sterilized by means which do not affect or denature the structure of a major portion of the type II collagen in the tissue, such as low-temperature processing, and formed into doses containing therapeutically effective levels of undenatured type II collagen, said levels being generally in the amount of at least about 0.01 gram and preferably from about 0.02 to about 0.5 grams of animal tissue in a dose. Being a natural product some variation from sample to sample is to be expected. These variations can be minimized by blending after comminution. The blending can be aided by analytical techniques which allow the measurement of the amount of undenatured type II collagen and other constituents.

Nonetheless, the present disclosure has found that by carefully forming the particles and sterilizing the type II collagen as discussed above, the undenatured type II collagen may be resistant to gastric acid and digestive enzymes in the stomach. Due to this sterilization process, the undenatured type II collagen also retains its 3-dimensional shape, preserving the bioactive epitope regions. Without wishing to be bound by theory, it is believed that the epitope regions contain the ability to induce oral tolerance as discussed above. Particularly epitope regions allow undenatured collagen to bind to the Peyer's Patches, which have the ability to induce oral tolerance processes.

In one aspect, the collagen composition is present in a serving of the processed animal food and/or treat in an amount from about 1 milligram to about 600 milligrams per gram of the processed animal food and/or treat. For instance, the collagen composition can be present in the processed animal food and/or treat in an amount of about 3 milligrams or more, such about 5 milligrams or more, such as about 7.5 milligrams or more, such as about 10 milligrams or more, such as about 12.5 milligrams or more, such as about 15 milligrams or more, such as about 25 milligrams or more, such as about 50 milligrams or more, such as about 75 milligrams or more, such as about 100 milligrams or more, such as about 125 milligrams or more, such about 150 milligrams or more, such as about 200 milligrams or more, such as about 250 milligrams or more, such as about 300 milligrams or more, such as about 350 milligrams or more, such as about 400 milligrams or more, such as about 450 milligrams or more, such as about 500 milligrams or more, such as about 550 milligrams or more, such as about 600 milligrams or more per gram of the processed animal food and/or treat. The total amount of collagen composition present in one gram of the processed animal food and/or treat is generally less than about 700 milligrams, such as less than about 600 milligrams, such as less than about 500 milligrams, such as less than about 400 milligrams, such as less than about 300 milligrams, such as less than about 250 milligrams, or any ranges or values therebetween. Additionally or alternatively, the collagen composition may be present in the processed animal food and/or treat in an amount of about 0.01% to about 10% by weight, such as about 0.1% to about 9%, such as about 0.25% to about 8%, such as about 0.5% to about 7.5%, such as about 0.75% to about 5% by weight of the processed animal food and/or treat composition, or any ranges or values therebetween. Furthermore, it should be understood that, in one aspect, the collagen composition may be a type II collagen composition, where substantially all of the collagen in the collagen composition is type II collagen.

In one aspect, undenatured type II collagen may form all, or substantially all, of the total type II collagen in the collagen composition, and therefore, may be present in the processed animal food and/or treat in the above discussed amounts. However, in one aspect, undenatured type II collagen may account for about 1% to about 95% of the total type II collagen and/or collagen composition, such as about 2.5% to about 75%, such as about 5% to about 50%, such as about 10% to about 40% of the total type II collagen or total collagen composition, or any ranges or values therebetween. Therefore, in one aspect, undenatured type II collagen may be present in the composition in an amount of 0.1 mg to about 100 mg, such as about 0.5 mg to about 75 mg, such as about 0.75 mg to about 50 mg, such as about 1 mg to about 30 mg per gram of processed animal food and/or treat, or any ranges or values therebetween.

Furthermore, in one aspect, the collagen composition may further include a preservative salt, such as potassium chloride. Thus, in one aspect, the total amounts of collagen composition discussed above may include type II collagen and/or undenatured type II collagen, alone or in combination with a further collagen, a preservative salt, or combinations thereof. In such as aspect, the total type II collagen, including native and undenatured type II collagen, may account for about 1% to about 99% of the collagen composition, such as about 2.5% to about 90%, such as about 5% to about 80%, such as about 7.5% to about 70%, such as about 10% to about 60%, such as about 15% to about 50%, such as about 20% to about 35%, or any ranges or values therebetween. Thus, in one aspect, the total amount of type II collagen, including native and undenatured type II collagen in the collagen composition may be from about 1 mg to about 1000 mg, such as about 2.5 mg to about 500 mg, such as about 5 mg to about 250 mg, such as about 7.5 mg to about 100 mg, such as about 10 mg to about 40 mg, or any ranges or values therebetween. Of course, in one aspect, no preservative salt is used.

Furthermore, in one aspect, when the type II collagen includes undenatured type II collagen, the undenatured type II collagen may have a large oxygen radical absorbance capacity (ORAC), as measured according to ORAC 6.0. Particularly, ORAC tests measure antioxidant scavenging activity against oxygen radicals that are known to be involved in the pathogenesis of aging and common disease, and consist of six types of ORAC assays that evaluate the antioxidant capacity of a material against primary reactive oxygen species, peroxyl radical, hydroxyl radical, superoxide anion, and peroxynitrite. Particularly, the ORAC assay includes introducing a reactive oxygen species (ROS) introducer to the assay system, where the ROS introducer triggers the release of a specific ROS which would degrade the probe and cause its emission wavelength or intensity to change. Thus, if the assay being tested includes an antioxidant, the antioxidant absorbs the ROS and preserves the probe from degradation. The degree of probe preservation indicates the antioxidant capacity of the material, and the results are expressed as μmol trolox equivalents (TE)/g of a tested material.

For example, an ORAC assay against peroxyl radical measures the antioxidant capacity of a sample to protect the fluorescent protein (fluorescein) from damage by a peroxyl radical which is generated from 2,2′ azobis(2 amidinopropane) dihydrochloride (AAPH). The ORAC assay against hydroxyl radical measures the antioxidant capacity of the sample to protect the fluorescent protein (fluorescein) from damage by a hydroxyl radical which is generated from reaction between cobalt and hydrogen peroxide. The ORAC assay against peroxynitrite measures the antioxidant capacity of the sample to protect Dihydrorhodamine-123 from damage by a peroxynitrite radical which is generated from 3-morpholinosydnonimine hydrochloride. The ORAC assay against superoxide measures the antioxidant capacity of the sample to protect hydroethidine from damage by a superoxide which is generated from xanthine oxidase. The ORAC assay against singlet oxygen measures the antioxidant capacity of the sample to protect hydroethidine from damage by single oxygen which is generated from a reaction between lithium molybdate and hydrogen peroxide. Finally, the ORAC assay against hypochlorite measures the antioxidant capacity of the sample to protect the fluorescent protein fluorescein from damage by the hypochlorite radical which is generated from sodium hypochlorite.

Thus, in one aspect, a collagen composition having an undenatured type II collagen according to the present disclosure may have a total ORAC of about 200 μmol TE/g or greater, such as about 250 μmol TE/g or greater, such as about 300 μmol TE/g or greater, such as about 350 μmol TE/g or greater, such as about 400 μmol TE/g or greater, such as about 450 μmol TE/g or greater, such as about 500 μmol TE/g or greater, such as about 550 μmol TE/g or greater, such as about 600 μmol TE/g or greater, such as about 700 μmol TE/g or greater, such as about 750 μmol TE/g or greater, such as about 800 μmol TE/g or greater, such as about 825 μmol TE/g or greater, up to about 1000 μmol TE/g, or any ranges or values therebetween.

Furthermore, in one aspect, a collagen composition having an undenatured type II collagen according to the present disclosure may have a ORAC against peroxyl radicals of about 1 μmol TE/g or greater, such as about 2.5 μmol TE/g or greater, such as about 5 μmol TE/g or greater, such as about 7.5 μmol TE/g or greater, such as about 10 μmol TE/g or greater, such as up to about 10.5 μmol TE/g or greater, up to about 50 μmol TE/g, or any ranges or values therebetween.

Similarly, in one aspect, a collagen composition having an undenatured type II collagen according to the present disclosure may have a ORAC against hydroxyl radicals of about 10 μmol TE/g or greater, such as about 15 μmol TE/g or greater, such as about 20 μmol TE/g or greater, such as about 25 μmol TE/g or greater, such as about 27.5 μmol TE/g or greater, such as about 30 μmol TE/g or greater, up to about 40 μmol TE/g, or any ranges or values therebetween.

Additionally or alternatively, in one aspect, a collagen composition having an undenatured type II collagen according to the present disclosure may have a ORAC against peroxynitrite of about 0.5 μmol TE/g or greater, such as about 1 μmol TE/g or greater, such as about 1.5 μmol TE/g or greater, such as about 2 μmol TE/g or greater, such as about 2.25 μmol TE/g or greater, up to about 5 μmol TE/g, or any ranges or values therebetween.

In one aspect, a collagen composition having an undenatured type II collagen according to the present disclosure may have a ORAC against singlet oxygen of about 500 μmol TE/g or greater, such as about 550 μmol TE/g or greater, such as about 600 μmol TE/g or greater, such as about 650 μmol TE/g or greater, such as about 700 μmol TE/g or greater, such as about 725 μmol TE/g or greater, up to about 1000 μmol TE/g, or any ranges or values therebetween.

Furthermore, in one aspect, a collagen composition having an undenatured type II collagen according to the present disclosure may have a ORAC against hypochlorite of about 25 μmol TE/g or greater, such as about 30 μmol TE/g or greater, such as about 35 μmol TE/g or greater, such as about 40 μmol TE/g or greater, such as about 45 μmol TE/g or greater, such as up to about 50 μmol TE/g or greater, up to about 75 μmol TE/g, or any ranges or values therebetween.

Furthermore, in one aspect, when the type II collagen includes undenatured type II collagen, the undenatured type II collagen may have a molecular weight of about 10,000 Daltons or more, such as about 15,000 Daltons or more, such as about 20,000 Daltons or more, such as about 25,000 Daltons or more, such as about 30,000 Daltons or more, such as about 35,000 Daltons or more, such as about 40,000 Daltons or more, such as about 45,000 Daltons or more, such as about 50,000 Daltons or more, such as about 55,000 Daltons or more, such as about 60,000 Daltons or more, such as about 65,000 Daltons or more, such as about 70,000 Daltons or more, such as about 75,000 Daltons or more, such as about 80,000 Daltons or more, such as about 85,000 Daltons or more, such as about 90,000 Daltons or more such as about 95,000 Daltons or more, such as about 100,000 Daltons or more, up to about 350,000 Daltons or less, or any ranges or values therebetween.

Moreover, it should be understood that, thus far, it has been contemplated that the undenatured collagen undergoes the processing with the processed animal food and/or treat. However, in one aspect, undenatured collagen may be incorporated into the animal food and/or treat both prior to processing and after processing. Therefore, in one aspect, undenatured collagen may be included in the animal food and/or treat prior to processing in an amount discussed above, and an amount of a collagen composition may be added to or incorporated into the animal food and/or treat after processing according to the amounts discussed above in regards to the collagen composition. The amount selected for pre and post processing may be the same or different, and may be based upon the total amount of undenatured that is desired to be present in the end composition.

While various aspects and benefits have been discussed, in one aspect, the collagen composition is incorporated into a suitable delivery form prior to incorporation into a dosage form as discussed below. In one aspect, the composition of the present disclosure may be included as an oil-in-water emulsion as a delivery form. Particularly, in one aspect, such an arrangement may allow one or more oil-soluble and/or one or more water-soluble active ingredients to be contained in the same delivery form. Alternatively, only oil-soluble components may be used (e.g. the Type II collagen), and the emulsion may be used to incorporate the composition into a water-based application.

Nonetheless, the oil-in-water emulsion may also contain at least one functional gum, such as gum arabic. Gum arabic, in general, is a complex mixture of glycoproteins and polysaccharides, including arabinose and galactose. Gum arabic is generally soluble in water and is edible. In some embodiments, the gum arabic may be comprised of a 100% modified gum arabic, such as Ticamulsion® A-2010 gum arabic powder. In certain embodiments, the gum arabic may be a mixture or blend of gum arabic and modified gum arabic. For example, in certain embodiments, the gum arabic may comprise Ticamulsion® 3020.

In certain aspects, the oil-in-water emulsion contains from about 10% to about 30% by weight of gum arabic. In some embodiments, the oil-in-water emulsion contains from about 15% to about 25% by weight of gum arabic. In some embodiments, the oil-in-water emulsion contains less than about 20% by weight of gum arabic, such as less than 15%, such as less than 10%, such at less than 5%.

The oil-in-water emulsion may also contain water. In certain aspects, the oil-in-water emulsion contains deionized water. Still, in certain aspects, the oil-in-water emulsion may contain any water suitable for ingestion by a mammal and incorporation into dietary supplements designed for ingestion by a mammal.

The amount of water incorporated into the oil-in-water emulsion can vary depending on the desired hygroscopic and water-soluble ingredients that are incorporated into the oil-in-water emulsion. In certain aspects, the oil-in-water emulsion may contain from about 5% to 35% by weight of water. In some embodiments, the oil-in-water emulsion may contain from about 10% to about 30% by weight of water. In some embodiments, the oil-in-water emulsion may contain from about 15% to about 20% by weight of water. In some embodiments, the oil-in-water emulsion may contain less than about 20% by weight of water, such as less than about 15% by weight of water, such as less than about 10% by weight of water.

In some aspects, the oil-in-water emulsion may contain one or more stabilizers or suspension promoting agents. For example, in certain aspects, the oil-in-water emulsion may contain one or more gum, such as gellan gum or xanthum gum. If included, the gellan gum or xanthum gum may be present in an amount of less than about 3.5% by weight of the oil-in-water emulsion, such as less than about 2.5% by weight, such as less than about 1.5% by weight, such as less than about 1.0% by weight, such as less than about 1.0% by weight.

In other aspects, the oil-in-water emulsion may contain one or more stabilizers such as silica. If included, silica may be present in an amount of less than about 2% by weight, such as less than about 1.5% by weight, such as less than about 1% by weight, such as less than about 0.5% by weight.

Furthermore, in one aspect, the oil-in-water emulsion may also contain one or more fat-soluble ingredients or nutrients. In certain aspects, the one or more fat-soluble ingredients or nutrients may be incorporated into the oil phase of the oil-in-water phase emulsion. Suitable fat-soluble ingredients include, but are not limited to retinol, vitamin E sourced from mixed tocopherols, beta carotene, ubiquinone, lecithin, sunflower lecithin, vitamin D, cannabinoids, hemp extracts, vitamin K, phosphatidyl choline, and combinations thereof.

In certain aspects, at least one or more fat-soluble ingredients may be incorporated in the oil-in-water emulsion in an amount of from about 0% by weight to about 50% by weight. For example, in some aspects, the oil-in-water emulsion contains less than about 50% by weight of one or more fat-soluble ingredients, such as less than about 40% by weight, such as less than about 30% by weight, such as less than about 20% by weight, such as less than about 10% by weight, such a less than about 5% by weight.

Moreover, in one aspect, the oil-in water emulsion may contain one or more additional antioxidants, in one or more of the water soluble phase, or the oil/fat soluble phase.

In some aspects, the oil-in-water emulsion disclosed herein may be used any suitable dosage form, such as gummy chewables, edible films, lozenges, liquid suspensions, syrups, lipid micelles, spray-dried dispersions, nanoparticles, and the like, which may also be incorporated into a further processed animal food and/or treat. Regardless of the dosage form, it should be clear that the dosage form, animal food and/or treat is processed at a temperature of at least 37° C. as discussed herein. Thus, it should be clear that a dosage form, animal food and/or treat that does not include a processing step as defined herein is not encompassed by the above definition. Thus, in one aspect, the dosage form, animal food and/or treat does not include a tablet or capsule.

The processed animal food and/or treat composition may include any suitable composition for consumption by the mammal. Such compositions include complete foods intended to supply the necessary dietary requirements for mammal or food supplements such as treats and snacks. The food composition may comprise pellet, a bar, a prepared food contained in a can, or any other functional food composition.

The processed animal food and/or treat composition of the present disclosure may further include one or more excipients as further additives in the composition. Exemplary but non-limiting excipients and/or additives include antiadherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, and waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, liquorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one aspect, the processed animal food and/or treat composition of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the composition in an amount sufficient to reduce the hydroscopic properties of the composition and/or prevent the composition from absorbing moisture. A stabilizer package may also be combined with the composition in order to improve the handling properties of the composition. For instance, the stabilizer package may allow the composition to have better flow properties, especially when in granular form.

In one aspect, the processed animal food and/or treat composition may be combined with a polymer binder in conjunction with a stabilizer package. In addition, a coating material may also be applied to the composition after the composition has been combined with the polymer binder and the stabilizer package. The coating material, for instance, may contain at least one fat. In accordance with the present disclosure, the above components can be added to any suitable pharmaceutical composition in addition to the composition of the present disclosure. For instance, the above components may be added to any pharmaceutical composition containing a carnitine or an amino acid.

The polymer binder and the stabilizer package may be combined with the processed animal food and/or treat composition in a manner that homogeneously incorporates the stabilizer package into the product. In one embodiment, for instance, the composition of the present disclosure is first combined with a polymer binder, such as through a spray dry process, and then combined with the stabilizer package. The polymer binder may comprise any suitable pharmaceutically acceptable polymer, such as film-forming polymers and/or polysaccharides. Particular examples of polymer binders that may be used in accordance with the present disclosure include starch, maltodextrin, gum arabic, arabinogalactan, gelatin, and mixtures thereof. In one embodiment, the polymer binder is added to the pharmaceutical composition in an amount of at least about 5% by weight, such as at least about 8% by weight, such as at least about 10% by weight, such as at least about 15% by weight. One or more polymer binders are present in the composition in an amount less than about 50% by weight, such as in an amount less than about 45% by weight, such as in an amount less than about 40% by weight, such as in an amount less than about 35% by weight, such as in an amount less than about 30% by weight.

In one embodiment, the polymer binder may comprise a starch, such as a modified starch. The starch, for instance, may be derived from corn or waxy maize. In one embodiment, the starch may comprise HI-CAP100 starch sold by National Starch and Chemical Company.

In an alternative embodiment, the polymer binder may comprise arabinogalactan. Arabinogalactan is a soluble polysaccharide that not only can serve as a polymer binder but may also provide other benefits. For instance, arabinogalactan may enhance the adaptive immune response in some circumstances. Arabinogalactan is described, for instance, in U.S. Pat. No. 8,784,844, which is incorporated herein by reference.

In one embodiment, larch arabinogalactan may be used as the polymer binder. Larch arabinogalactan is a highly branched polysaccharide that is composed of galactose units and arabinose units in the approximate ratio of 6:1. Larch arabinogalactan is extracted from large trees. The polysaccharide has a galactan backbone with side chains of galactose and arabinose. Arabinogalactan is commercially available from Lonza Ltd.

Once the polymer binder is combined with the composition such as through a spray dry process, the resulting mixture can then be combined with a stabilizer package. In one embodiment, the stabilizer package comprises oxide particles in combination with a salt of a carboxylic acid. In one particular embodiment, the stabilizer package may comprise a dry product, such as a powder or granular product that is combined with the composition and polymer binder. The combination of oxide particles and a salt of a carboxylic acid have been found to provide numerous advantages and benefits when combined with the composition. For instance, the stabilizer package has been found to stabilize the composition and make the composition less hydroscopic. The composition is also easier to handle and, when in granular form, produces a free-flowing product.

The oxide particles that may be added to the processed animal food and/or treat composition may comprise silica. For instance, the oxide particles may comprise precipitated silica particles. The silica particles may have a particle size (d50, laser defraction following ISO Test 13320) of less than about 55 microns, such as less than about 40 microns, such as less than about 30 microns, such as less than about 25 microns, such as less than about 20 microns, such as less than about 15 microns, such as less than about 12 microns, such as less than about 10 microns, such as less than about 8 microns, such as less than about 6 microns, such as less than about 4 microns, such as less than about 2 microns, such as less than about 1 micron. The particle size is typically greater than about 0.5 microns, such as greater than about 1 micron. The particles may have a specific surface area (ISO Test 9277) of greater than about 120 m2/g, such as greater than about 130 m2/g, such as greater than about 150 m2/g, such as greater than about 170 m2/g, such as greater than about 200 m2/g, such as greater than about 220 m2/g. The specific surface area is generally less than about 500 m2/g. The oxide particles, such as the silica particles, can be present in the pharmaceutical composition in an amount greater than about 0.01% by weight, such as in an amount greater than about 0.05% by weight, such as in an amount greater than about 0.1% by weight. The oxide particles are generally present in an amount less than 5% by weight, such as in an amount less than about 2% by weight, such as in an amount less than about 1.5% by weight, such as in an amount less than 0.5% by weight.

In addition to the oxide particles, the stabilizer package may also include a salt of a carboxylic acid. The salt of a carboxylic acid may comprise a salt of a fatty acid. The fatty acid, for instance, may have a carbon chain length of from about 6 carbon atoms to about 40 carbon atoms, such as from about 12 carbon atoms to about 28 carbon atoms. In one embodiment, the salt of the carboxylic acid may comprise a stearate salt. The stearate salts that may be used include calcium stearate, sodium stearate, magnesium stearate, mixtures thereof, and the like. In one embodiment, the salts of the carboxylic acid may include both hydrophilic groups and hydrophobic groups. The salt of the carboxylic acid may be present in the composition in an amount greater than about 0.5% by weight, such as in an amount greater than about 1% by weight, such as in an amount greater than about 1.5% by weight. The salt of the carboxylic acid is generally present in an amount less than about 5% by weight, such as in an amount less than about 4% by weight, such as in an amount less than about 3% by weight.

In addition to the polymer binder and the stabilizer package, the composition may include various other components and ingredients. In one embodiment, for instance, the composition may contain a citric acid ester, such as a citric acid ester of a mono and/or diglyceride of a fatty acid. The composition may also contain a lecithin, such as a lecithin obtained from rapeseed, sunflower, and the like. The above components can be present in the composition in relatively minor amounts, such as less than about 2% by weight, such as less than about 1.5% by weight, such as less than about 1% by weight. The above components are generally present in an amount greater than about 0.05% by weight, such as in an amount greater than about 0.1% by weight.

Furthermore, in one aspect, the processed animal food and/or treat may be formulated into a food or treat for sports or daily nutritional purposes. In such an aspect, the processed animal food and/or treat may further include at least one vitamin, such as at least one of vitamin B, vitamin C, and vitamin E. Vitamins may be contained in the processed animal food and/or treat in an amount of from about 50 μg/g of supplement to about 5000 μg/g, such as about 100 μg/g to about 4500, such as about 250 μg/g to about 4000 μg/g, such as about 400 μg/g to about 3500 μg/g, or any ranges or values therebetween. The above ranges may be for any one vitamin alone or a total amount of all vitamins. In one aspect, vitamin E is present in processed animal food and/or treat in an amount of about 100 μg/g to about 1000 μg/g, such as about 250 μg/g to about 750 μg/g, such as about 400 μg/g to about 600 μg/g, or any ranges or values therebetween. In another aspect, vitamin C is present in processed animal food and/or treat in an amount of about 1000 μg/g to about 5000 μg/g, such as about 2000 μg/g to about 4000 μg/g, such as about 3000 μg/g to about 3750 μg/g, or any ranges or values therebetween.

Furthermore, in an aspect, the processed animal food and/or treat contains at least one mineral, such as at least one of potassium magnesium, zinc, or calcium. Minerals may be contained in the processed animal food and/or treat in an amount of from about 1 mg/g to about 50 mg/g, such as about 2.5 mg/g to about 45 mg/g, such as about 5 mg/g to about 40 mg/g, or any ranges or values therebetween. The above ranges may be for any one mineral or a total amount of one mineral. In one aspect, the processed animal food and/or treat contains potassium in an amount of about 9.5 mg/g to about 12 mg/g, such as about 9.75 mg/g to about 11.5 mg/g, such as about 10 mg/g to about 11 mg/g, or any ranges or values therebetween. Similarly, in one aspect, the processed animal food and/or treat contains magnesium in an amount of about 1 mg/g to about 10 mg/g, such as about 2.5 mg/g to about 7.5 mg/g, such as about 4 mg/g to about 6 mg/g, or any ranges or values therebetween. Furthermore, in one aspect, the processed animal food and/or treat contains calcium in an amount of about 1 mg/g to about 50 mg/g, such as about 2.5 mg/g to about 47.5 mg/g, such as about 5 mg/g to about 45 mg/g, such as about 10 mg/g to ab out 40 mg/g, such as about 20 mg/g to about 37.5 mg/g, such as about 30 mg/g to about 35 mg/g, or any ranges or values therebetween.

Additionally, the processed animal food and/or treat may further include at least one additive that enhances sports performance or that contributes to reducing oxidative stress. For instance, in one aspect, an additive may be one or more of curcumin, spirulina, astaxanthin, carnitine, or other carotenoids. Furthermore, in one aspect, the present disclosure may include one or more microalgae with a high superoxide dismutase (SOD) and/or ORAC level. Particularly, such microalgae may further help to reduce oxidative stress, and may contribute further anti-inflammatory properties and protection against infections, including improvement in immune health. Moreover, in one aspect, an additive may include one or more probiotics.

Furthermore, in one aspect, the processed animal food and/or treat may be formulated to include other components for daily nutrition such as an additional protein source, one or more grains, dietary fibers, starches, fruits, vegetables, or combinations thereof that are suitable for ingestion and dietary support of non-human mammals. In one aspect, the additional protein source may be plant or animal based, or may be a combination thereof.

Nonetheless, in one aspect, for instance, the processed animal food and/or treat of the present disclosure is particularly formulated to improve joint health, muscle health, cartilage heath, bone health, or combinations thereof. For instance, the processed animal food and/or treat can be used to treat non-arthritic joint pain, joint discomfort in healthy mammals, lack of joint flexibility in healthy mammals, muscle soreness in healthy mammals, or lack of fitness in healthy mammals. In addition, the processed animal food and/or treat of the present disclosure can improve immune health, bone health, or brain health, and may also improve triglyceride and/or cholesterol levels in a healthy mammal and/or a mammal that is regularly undergoing physical activity and/or intense physical activity. Furthermore, the processed animal food and/or treat according to the present disclosure may also improve joint health, bone health, muscle health and soreness, and cartilage health that is caused by age related decline. Thus, in one aspect, the processed animal food and/or treat may also include one or more additional joint supplements such as hydroxy citric acid, glucosamine, chondroitin, or the like, or combinations thereof, and/or an enhancer of collagen absorption, such as vitamin c, in addition to the undenatured collagen.

However, in one aspect, it should be understood that no additional joint supplements are necessary in the processed food and/or treat. For instance, in one aspect, the processed animal food and/or treat may include ingredients for daily nutrition of a non-human mammal, include high levels of crude protein(s), fats, and dietary fibers. In one aspect, the crude protein(s) may include one or more protein meal, such as a mealed protein produce formed from one or more animal protein sources, which may include bone, organ, cartilage, and skin of a mammal.

For instance, in one aspect, a non-human processed food and/or treat may include about 18 wt. % to about 40 wt. % crude protein, about 4 wt. % to about 30 wt. % fat, and about 2 wt. % to about 20 wt. % total dietary fiber. In another aspect, the processed food and/or treat may be a low-fat diet to promote weight loss. A typical low-fat diet may contain about 18 wt. % to about 22 wt. % protein, about 8 wt. % to about 10 wt. % fat, and about 1 wt. % to about 3 wt. % crude fiber. Particularly, it should be clear to one having skill in the art that appropriate levels for a non-human mammal are not consistent for levels necessary or recommended for human nutrition.

Moreover, the processed animal food and/or treat may be suitable for administration to any non-human mammal. For instance, the mammal may be canine, bovine, feline, or equine. The composition can be fed to a non-human mammal of any age such as from parturition through the adult life in the mammal. In various embodiments the mammal may be a dog, a cat, a horse, a pig, a sheep, or a cow. In many embodiments, the mammal can be in early to late adulthood. For instance, the active mammal may have an age that is at least 10%, such as least 15%, such as least 20%, such as least 25%, such as least 30%, such as least 35%, such as least 40%, such as least 45%, such as least 50%, such as least 55%, such as least 60%, such as least 65%, such as least 70%, such as least 75%, such as least 85%, such as least 90%, such as least 95% of its expected life span. The mammal may have an age such that it is less than about 95%, such as less than about 90%, such as less than about 85%, such as less than about 80%, such as less than about 75%, such as less than about 70%, such as less than about 65%, such as less than about 60%, such as less than about 55%, such as less than about 50%, such as less than about 45%, such as less than about 40%, such as less than about 35%, such as less than about 30%, such as less than about 25%, such as less than about 20%, such as less than about 15%, such as less than about 10% of its expected life span. A determination of life span may be based on actuarial tables, calculations, or the like.

Nonetheless, certain embodiments of the present disclosure may be better understood according to the following examples, which are intended to be non-limiting and exemplary in nature.

### Example 1

Plant and Animal Based Dry Foods

A batch of animal based dry food was formed by mixing chicken breast, dried eggs, whole grain wheat flour, oatmeal, corn meal, oat bran, canola oil and water. A batch of vegetable based dry food was formed by mixing natural balance vegetable dry dog formula with dried eggs, whole grain wheat flour, oatmeal, corn meal, oat bran, canola oil and water. Both the plant and animal based dry food were spiked with UC-II® brand Undenatured Type II Collagen as shown in Table 1 below, and were then formed, cooked in an oven at 120°, and dried to 10% moisture. After cooking and drying, the samples were tested for undenatured collagen retained post processing and cooking, as reflected in Table 1 as % Recovery. Animal based foods are noted as “A” samples and plant based foods are noted as “P” samples.

TABLE I
Undenatured Type II Collagen Recovery

| | | | | | mg | |
|---|---|---|---|---|---|---|
| | | | mg | | undenatured | |
| | | | undenatured | | Type II | |
| | mg UCII | mg Type II | Type II | mg Type II | collagen per | |
| | added per 3 | Collagen per | Collagen per | collagen per | gram of | |
| | grams of | gram of solid | gram of solid | gr solid after | solid after | % |
| Sample | solid food | food | food | formation | formation | Recovery |
| Control A | 0 | | | | 0.01 | |
| Control P | 0 | | | | 0.35 | |
| A | 10 | 2.56 | 0.8 | 3.6 | 1.12 | 139.7 |
| A | 60 | 15.4 | 4.81 | 9.3 | 7.23 | 150.3 |
| A | 200 | 51.4 | 16 | 64.8 | 20.89 | 130.6 |
| P | 10 | 2.56 | 0.8 | 2.7 | 0.93 | 116 |
| P | 60 | 15.4 | 4.81 | 19.2 | 4.03 | 83.8 |
| P | 200 | 51.4 | 16 | 46.8 | 20.16 | 126 |

As shown in Table I, both plant and animal based foods exhibited excellent recovery of undenatured type II collagen after formation and cooking, even at moisture levels typical for pet food. The recoveries of greater than 100% are at least in part due to endogenous undenatured type II collagen contained in the chicken used in the animal based food, but may also be partly due to the moisture content of the food. In regards to plant based food, without wishing to be bound by theory, it is believed that an increase in undenatured type II collagen recovery may be due at least in part to interactions with other nutrients contained in the food. Additionally, due to the small variability and lab assay potential, the value may be considered to be essentially 100% recovery for plant based samples reporting greater than 100% recovery.

### Example 2

Extruded Pet Food

Animal based pet food was formed according to example 1, except that the pet food was extruded into the desired shape prior to cooking. UC-II® brand Undenatured Type II Collagen was added prior to forming injection molded extruded powder mixture as show in Table 2 below.

TABLE 2
Undenatured Type II Collagen Recovery

| | Amount of | Avg Amount of | | |
|---|---|---|---|---|
| | Undenatured | Undenatured | | Wt. % |
| | Collagen | Collagen | Avg % | Undenatured |
| | Determined by | Determined by | Recovery | Type II |
| Sample | Elisa (mg) | Elisa (mg) | *** | Collagen |
| Undenatured | 329.9 | 329.9 | n/a | 11.00* |
| Type II | | | (100%) | |
| Collagen | | | | |
| Injection | 2.87 | 2.82 | 70.5 | 5.64** |
| Molded | | | | |
| Powder 1 | | | | |
| Injection | 2.76 | | | |
| Molded | | | | |
| Powder 2 | | | | |

*based upon 3 grams of Undenatured Type II Collagen subjected to extraction
**based on 10 milligrams of Undenatured Type II Collagen per gram of Powder Mixture; a total of 50 milligrams Undenatured Type II Collagen in each 5 gram extraction
*** at theoretical, each extract sample contains 4.0 mg undenatured collagen

### Example 3

Extruded Dental Sticks

Potato Flour, Coconut Glycerin, Natural Chicken Flavor, Pea Protein, Canola Oil, Powdered Cellulose, Coconut Oil, Dried Cultured Skim Milk, Citric Acid (Preservative), Sodium Hexametaphosphate, Vanilla, Natural Mint Flavor, Zinc Propionate, Mixed Tocopherols (Preservative), Green Tea Extract, and Rosemary Extract were combined to form the dental stick mixture. The dental stick mixture was then extruded and baked as discussed above in regards to the formation of pet food, except the extruded product was shaped into a dental stick. The dental sticks contained UC-II® brand Undenatured Type II Collagen as shown in Table 3 below. Control dental sticks were formed in the same manner as the example except without any added UC-II® brand Undenatured Type II Collagen. Both the example and control and were subjected to recovery testing, the results of which are shown in Table 3.

TABLE 3
Undenatured Type II Collagen Recovery

| | Amount of | AVG Amount of | | | |
|---|---|---|---|---|---|
| | Undenatured | Undenatured | | Wt. % | Avg Wt. % |
| | Type II Collagen | Type II Collagen | | Undenatured | Undenatured |
| | Determined by | Determined by | Avg % | Type II | Type II |
| Sample | ELISA (mg) | ELISA (mg) | Recovery | Collagen | Collagen |
| Undenatured | 360.97 | 360.97 | N/A | 12.03 | 12.03 |
| Type II | | | (100%) | | |
| Collagen | | | | | |
| Dental Stick | Not detected | N/A | N/A | 0 | 0 |
| Control 1 | | | | | |
| Dental Stick | 0.02 | | | 0 | |
| Control 2 | | | | | |
| Dental Stick 1 | 23.85 | 22.14 | 74.8 | 6.34 | 5.98 |
| Dental Stick 2 | 1.6 | | | 5.61 | |

### Example 4

### Undenatured Type II Collagen Formulated Brown Rice Flour

Brown rice flour was formulated containing varying levels of UC-II® brand Undenatured Type II Collagen. It was found that the formulated brown rice flour was stable (e.g. maintained levels of undenatured type II collagen) and suitable for use in preparing processed foods such as pet food and/or treats. Brown rice was mixed with UC-II® brand Undenatured Type II Collagen at rations of UC-II® brand Undenatured Type II Collagen to Brown rice flour of 80:20, 50:50, and 20:80. After mixing to incorporation (e.g. no separation) the samples were free-dried and analyzed to determine the levels of undenatured type II collagen remaining in the formulated brown rice, the results of which are shown in table 4.

TABLE 4
ELISA for Undenatured Type II Collagen

| | Total Amount | Average Amount | Average Wt. % |
|---|---|---|---|
| | Undenatured | Undenatured | Undenatured |
| | Type II | Type II | Type II |
| Sample | Collagen (mg) | Collagen (mg) | Collagen |
| Undenatured Type | 254.9 | 254.9 | 8.5 |
| II Collagen (UTIIC) | | | |
| Brown Rice | 0 | 0 | 0 |
| Flour(BRF) Control | | | |
| 1 | | | |
| Brown Rice Flour | 0 | | |
| (BRF) Control 2 | | | |
| 80:20 UTIIC:BRF 1 | 137.74 | 146.2 | 4.9 |
| 80:20 UTIIC:BRF 2 | 154.65 | | |
| 50:50 UTIIC:BRF 1 | 129.56 | 122.36 | 4.1 |
| 50:50 UTIIC:BRF 2 | 115.16 | | |
| 20:80 UTIIC:BRF 1 | 48.43 | 49.54 | 1.6 |
| 20:80 UTIIC:BRF 2 | 50.66 | | |

### Example 5

Effect of Pet Food and/or Treats Containing Undenatured Collagen on Inflammation and Joint Pain in Exercised Labrador Retrievers

Objective: the objective of this study is to evaluate the effectiveness of Undenatured Type II Collagen on the mediation of inflammation and joint pain in Labrador retriever dogs during and after endurance running exercise. UC-II® brand Undenatured Type II Collagen is thought to decrease inflammation and joint pain when given orally. This will be evidenced by alterations in inflammatory and general health indicators including: 1) improvement or comparative maintenance of gait parameters via pressure walkway, 2) improvement or comparative maintenance of joint health and inflammatory biomarkers (CK, COMP. IL-6, and N:L ratio), 3) improvement or comparative maintenance of objective lameness scores, and 4) improved activity during running exercise.

Animals and Housing: forty healthy Labrador retrievers (20 male, 20 female) will be utilized during the trial. Body weights will range from 22-38 kg with a mean of 30 kg. All dogs will be in fit condition when beginning the study, with body composition scores of 3 to 6. The age of the dogs will range from 5 to 11 years with a mean of 7.5 years. All dogs will be housed individually overnight and will be aired in social groups in outside yards for 6 hours per day, dependent upon weather and testing status. All dogs will be fed their assigned diets and treatment once daily in the morning and have free access to automatic waterers at all times. All dogs will be up to date on vaccinations and receive monthly prophylactic heartworm and parasite prevention.

Running exercise: all dogs will begin an outdoor endurance running program after a two week loading period. The running regimen will be prescribed as follows: weeks 3-5: 2 miles twice weekly, Weeks 6-8: 4 miles twice weekly, Weeks 9-11: 5 miles twice weekly, Week 12: 2 miles twice weekly, Week 13: 10 miles once weekly. While running, all dogs will wear Article® accelerometer collars to quantify activity intensity, and Garmin® GPS collars to quantify distance and moving speed. All dogs will run in groups alongside an all-terrain vehicle and will be free to run, stop, swim, play, etc. Every effort will be made to keep the dogs active for the duration of each run, but any dog that refuses will be allowed to return to the kennel.

Gait analysis: all days will have gait analysis performed using a pressure walkway (Gait4Dogs) at baseline, 24 h prior to the first 3 mile run, 24 and 48 h after the first 3 mile run, 24 h prior to the 10 mile run, and 24 and 48 h after the 10 mile run. Each dog will be passed over the walkway 6-12 times at each timepoint to obtain at least 3-4 valid walkway samples for analysis. A variety of temporal, pressure, and spatial parameters per limb for each valid walk will be collected. Calculations will also be performed to provide all limb, front limb, and hind limb, averages as well as left:right front and hind limb symmetry ratios for each parameter.

Biological sampling: blood samples will be taken at baseline, 24 h prior to the first 3 mile run, 24 h after the first 3 mile run, 24 h prior to the 10 mile run, and 24 h after the 10 mile run for biomarker and hematology purposes. Biomarkers creatine kinase (CK), interleukin-6 (IL-6), and cartilage oligomeric matrix protein (COMP) will be evaluated using commercially available ELISA kits. Hematology will be analyzed using an automatic hematology machine (Abaxis HM5).

Pain assessment: pain will be assessed by subjective analysis using the LOAD questionnaire. Three trained technicians will observe the dogs in their kennels and outside and will score the dogs based on the questionnaire items. Data from all three technicians will be compiled and analyzed.

Statistical methods: JMP 14.0 will be used to create a mixed model to compare the difference and change of food intake, body weights, blood biomarkers, hematology, running activity, running moving speed, and gait analyses between treatments. Sex will be analyzed as a fixed effect. Results will be considered significant if a p-value of 0.05 or less is obtained.

Experimental design: Forty healthy Labrador retrievers (20 male/20 female) will be utilized in a trial to evaluate the effectiveness of Undenatured Type II Collagen on the mediation of inflammation and joint pain during and after exercise. Dogs will be sorted by age, body weight, and genetics into two equalized treatment groups. The treatment group will receive a 40 mg capsule of the test supplement UC-II® brand Undenatured Type II Collagen daily, and the placebo group will receive a capsule of maltodextrin daily. After a two week acclimation/loading period, each dog will begin an outdoor endurance exercise running regimen increasing in distance incrementally over 11 weeks. Each dog will have blood samples taken, gait analysis performed, and pain assessment performed at baseline prior to beginning the loading period, before and after the first 3 mile run, and before and after the 10 mile run.

### Example 6

### Effect of Undenatured Type II Collagen (UTIIC) on Sodium Monoiodoacetate Induced Osteoarthritis (OA) in Rats

Male Wistar rats were divided into 3 groups: (i) Control; (ii) MIA-induced rat treated with vehicle; (iii) MIA-induced rats treated with UC-II® brand Undenatured Type II Collagen (4 mg/kg). OA was induced in male Wistar rats by intra-articular injection of sodium monoiodoacetate (MIA: 1 mg). Treatment was started a week before injection with MIA and lasted 30 days. Biomarker testing was conducted prior to 24 days post MIA. The results of the metabolic marker testing is shown in Table 5, and inflammatory markers are shown in Table 6.

TABLE 5

| Test | Control | MIS | MIA + UTIIC | P-Value |
|---|---|---|---|---|
| GLU (mg/dL) | 108.43 ± 6.92 | 113.86 ± 10.81 | 109.86 ± 5.67 | 0.446 |
| CR (mg/dL) | 0.44 ± 0.09 | 0.41 ± 0.06 | 0.41 ± 0.09 | 0.801 |
| BUN (mg/dL) | 22.84 ± 2.15 | 23.27 ± 3.66 | 23.47 ± 3.36 | 0.929 |
| TP (g/dL) | 6.84 ± 0.41 | 6.73 ± 0.45 | 7.00 ± 0.2 | 0.404 |
| ALB (g/dL) | 3.63 ± 0.25 | 3.4 ± 0.14 | 3.54 ± 0.05 | 0.061 |
| GLOB (g/dL) | 3.16 ± 0.24 | 3.29 ± 0.33 | 3.39 ± 0.13 | 0.247 |
| ALT (U/L) | 67.86 ± 7.27 | 70.14 ± 5.9 | 70.43 ± 6.19 | 0.722 |
| AST (U/L) | 111.71 ± 12.18 | 115.29 ± 7.5 | 109.43 ± 8.77 | 0.534 |
| TBIL (MG/dL) | 0.2 ± 0.01 | 0.19 ± 0.03 | 0.21 ± 0.02 | 0.166 |

MIA: Monosodium iodoacetate
BUN: Blood Urine Nitrogen
TP: Total Protein
ALT: Alanine aminotransferase
AST: Aspartate aminotransferase
TBIL: Total Bilirubin
CR: Creatine
GLU: Glucose
ALB: Albumin
GLOB: Globulin

TABLE 6

| | | | | P- |
|---|---|---|---|---|
| Marker | Control | MIA | MIA + UTIIC | Value |
| IL-1β | 13.26 ± 5.36 | 42.4 ± 2.51 | 39.14 ± 2.77 | 0.001 |
| (pg/mL) | | | | |
| IL-6 (pg/mL) | 6.7 ± 0.48 | 44.06 ± 4.1 | 36.14 ± 1.67 | 0.001 |
| TNF-α | 19.01 ± 2.42 | 73.08 ± 5.14 | 54.19 ± 4.3 | 0.001 |
| (pg/mL) | | | | |
| COMP | 5.92 ± 0.62 | 32.2 ± 2.35 | 26.92 ± 2.01 | 0.001 |
| (ng/mL) | | | | |
| CRP (mg/L) | 1.56 ± 0.1 | 11.64 ± 1.42 | 7.87 ± 0.82 | 0.001 |
| PGE2 (pg/mL) | 248.3 ± 28.87 | 668.96 ± 42.11 | 537.81 ± 24.56 | 0.001 |
| OCN (μg/mL) | 49.73 ± 2.71 | 17.91 ± 1.94 | 23.08 ± 2.05 | 0.001 |

MIA: Monosodium iodoacetate
TNF-α: tumor necrosis factor α
COMP: cartilage oligomeric matrix protein
PGE2: prostaglandin E2
IL-1β: interleukin-1β
IL-6: interleukin-6
CRP: c-reactive protein
OCN: osteocalcin

As shown in Tables 6, Undenatured Type II Collagen was shown to reduce MIA-induced Kellgren-Lawrence scoring (53.3%) of OA by reduction of articular cartilage damage in rats (P<0.05). Suppression of pro-inflammatory cytokines [IL-1β (7.8%), IL-6 (18.0%), TNF-α (25.9%), COMP (16.4%), CRP (32.4%)] production was also found after Undenatured Type II Collagen treatment (P<0.0001). Undenatured Type II Collagen was also found to inhibit the production of PGE2 (19.6%), and the expression of IL-1β, IL-6, TNF-α, COX-2, MCP-1, NF-κB, MMP-3, RANKL (P<0.001). The levels of Col-1 and OPG were increased in MIA-induced OA rats (P<0.001). Additionally, it was found that the MIA+UCII mice exhibited a higher superoxide dismutase level (44.19±2.15 U/mL) compared to the MIA mice (37.98±3.19). Therefore, it is further clear that undenatured type II collagen in undenatured form is beneficial to mammals, such as in regards to joint health, cartilage health, bone health, and muscle health, to name a few.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A processed animal food and/or treat composition comprising:
an undenatured type II collagen;
wherein the processed animal food and/or treat is processed at a temperature of about 37° C. or greater.

2. The composition as defined in claim 1, wherein the composition comprises a processed animal food.

3. The composition as defined in claim 1, wherein the composition comprises a processed animal treat.

4. The composition as defined in claim 1, wherein the undenatured type II collagen is incorporated into the processed animal food and/or treat composition as part of a collagen composition comprising one or more different types of collagen in addition to the undenatured type II collagen.

5. The composition as defined in claim 1, wherein the one or more different types of collagen include native type II collagen, collagen peptides, or a mixture thereof.

6. The composition as defined in claim 1, wherein an amount of undenatured type II collagen is incorporated into the composition prior to processing and at least about 30% or more of the undenatured type II collagen is recovered after processing.

7. The composition as defined in claim 6, wherein 60% or more of the undenatured type II collagen is recovered after processing.

8. The composition as defined in claim 1, wherein the processed animal food and/or treat undergoes processing that includes withstanding a temperature of about 40° C. or greater.

9. The composition as defined in claim 1, wherein the processing lasts from 6 seconds to about 2 hours.

10. The composition as defined in claim 1, wherein the animal food and/or treat includes one or more of a protein source, a grain, a flavoring, or a coloring.

11. The composition as defined in claim 1, wherein the processed animal food and/or treat includes about 15 wt. % to about 40 wt. % crude protein, about 5 wt. % to 30 wt. % fat, and about 2 wt. % to about 20 wt. % dietary fiber.

12. The composition as defined in claim 1, wherein the processed animal food and/or treat includes a processed animal meal.

13. A method of forming a processed animal food and/or treat comprising
combining an undenatured type II collagen with at least one animal food and/or treat component, and
processing the undenatured type II collagen and at least one animal food and/or treat component at a temperature of about 37° C. or greater,
wherein at least about 30% or more of the undenatured type II collagen is recovered in the processed animal food and/or treat after processing as compared to the amount of undenatured type II collagen prior to processing.

14. The method of claim 13, wherein the processing includes subjecting the undenatured type II collagen and at least one animal food and/or treat component to a temperature of about 40° C.

15. The method of claim 13, wherein the processing includes subjecting the undenatured type II collagen and at least one animal food and/or treat component to a temperature of about 37° C. for at least about 10 minutes.

16. The method of claim 13, wherein the processing includes subjecting the undenatured type II collagen and at least one animal food and/or treat component to a temperature of about 100° C. for at least about 1 minute.

17. The method of claim 13, wherein the processing includes a pelletizing process.

18. The method of claim 13, wherein the processing further includes extruding the composition.

19. The method of claim 13, wherein the processing further comprises injection molding the composition.

20. A method of improving one or more of joint health, cartilage health muscle health, bone health, skin health, inflammation markers, or fitness comprising, administering to a non-human mammal an effective amount of a processed animal food and/or treat according to claim 1.
